(19) United States
(12) Patent Application Publication
(10) Pub. No.: US 2017/0095226 A1
(43) Pub. Date: Apr. 6, 2017

(54) ULTRASONIC DIAGNOSTIC APPARATUS AND MEDICAL IMAGE DIAGNOSTIC APPARATUS

(71) Applicant: Toshiba Medical Systems Corporation, Otawara-shi (JP)

(72) Inventors: Go TANAKA, Otawara (JP); Shinichi HASHIMOTO, Otawara (JP); Takayuki GUNJI, Otawara (JP); Osamu NAKAJIMA, Otawara (JP); Eiji GOTO, Utsunomiya (JP); Tomokazu FUJII, Nasushiobara (JP)

(73) Assignee: Toshiba Medical Systems Corporation, Otawara-shi (JP)

(21) Appl. No.: 15/283,832

(22) Filed: Oct. 3, 2016

(30) Foreign Application Priority Data
Oct. 5, 2015 (JP) 2015-197881

(51) Int. Cl. A61B 8/08; A61B 8/00

(52) U.S. Cl. CPC A61B 8/0841; A61B 8/463; A61B 8/466; A61B 8/5223; A61B 2017/3413

(57) ABSTRACT

An ultrasonic diagnostic apparatus according to an embodiment includes an acquiring function, a calculating function, and a correcting function. The acquiring function acquires first positional information indicating the position of a puncture needle in a space from which an ultrasonic image is acquired and second positional information indicating the position of the puncture needle included in the ultrasonic image. The calculating function calculates a bend in the puncture needle based on the first positional information and the second positional information. The correcting function corrects the position of information indicating the puncture needle with respect to the ultrasonic image assumed based on the first positional information.

### CROSS-REFERENCE TO RELATED APPLICATIONS

This application is based upon and claims the benefit of priority from Japanese Patent Application No. 2015-197881, filed on Oct. 5, 2015; the entire contents of which are incorporated herein by reference.

### FIELD

Embodiments described herein relate generally to an ultrasonic diagnostic apparatus and a medical image diagnostic apparatus.

### BACKGROUND

To facilitate observation of a state in a subject's body, conventionally widely used are ultrasonic diagnostic apparatuses that transmit ultrasonic waves from the surface of the body to the inside thereof and display an ultrasonic image based on reflected waves. Ultrasonic diagnostic apparatuses are used to perform a puncture in biopsies, radio-frequency ablation (RFA), and treatment using irreversible electroporation (IRE), for example, because they can display an ultrasonic image on a monitor substantially in real-time.

In a biopsy, for example, a doctor inserts a puncture needle into a lesion while checking the position of the needle point of the puncture needle and/or the position of the lesion in an ultrasonic image to obtain a tissue from the lesion. In RFA or treatment using IRE, a doctor inserts a puncture needle into a lesion while checking the position of the needle point and/or the position of the lesion and outputs radio-frequency waves from the puncture needle to cauterize the lesion. The procedures described above are performed with a guideline that guides insertion of the puncture needle displayed on the ultrasonic image.

### BRIEF DESCRIPTION OF THE DRAWINGS

FIG. 1 is a block diagram of an exemplary configuration of an ultrasonic diagnostic apparatus according to a first embodiment;

FIG. 2A is a diagram for explaining an example of a position detection system according to the first embodiment;

FIG. 2B is a diagram of an example of a guideline for a puncture needle according to the first embodiment;

FIG. 2C is a diagram for explaining setting of a target and markers according to the first embodiment;

FIG. 3A is a diagram for explaining an example of calculation of a bend in the puncture needle according to the first embodiment;

FIG. 3B is another diagram for explaining the example of calculation of an bend in the puncture needle according to the first embodiment;

FIG. 4 is a diagram for explaining another example of calculation of a bend in the puncture needle according to the first embodiment;

FIG. 5A is a diagram of an example of display information according to the first embodiment;

FIG. 5B is another diagram of the example of display information according to the first embodiment;

FIG. 6A is still another diagram of the example of display information according to the first embodiment;

FIG. 6B is still another diagram of the example of display information according to the first embodiment;

FIG. 7 is still another diagram of the example of display information according to the first embodiment;

FIG. 8 is still another diagram of the example of display information according to the first embodiment;

FIG. 9 is still another diagram of the example of display information according to the first embodiment;

FIG. 10A is a diagram of an example of display information according to the first embodiment;

FIG. 10B is another diagram of the example of display information according to the first embodiment;

FIG. 11A is still another diagram of the example of display information according to the first embodiment;

FIG. 11B is still another diagram of the example of display information according to the first embodiment;

FIG. 12 is a flowchart for explaining exemplary processing performed by the ultrasonic diagnostic apparatus according to the first embodiment;

FIG. 13 is a diagram for explaining an example of two-dimensional distance calculation according to a second embodiment;

FIG. 14A is a diagram for explaining an example of three-dimensional distance calculation according to the second embodiment;

FIG. 14B is another diagram for explaining the example of three-dimensional distance calculation according to the second embodiment; and

FIG. 14C is still another diagram for explaining the example of three-dimensional distance calculation according to the second embodiment.

### DETAILED DESCRIPTION

According to an embodiment, an ultrasonic diagnostic apparatus includes processing circuitry. The processing circuitry is configured to acquire first positional information indicating a position of a puncture needle in a space from which an ultrasonic image is acquired and second positional information indicating a position of the puncture needle included in the ultrasonic image. The processing circuitry is configured to calculate a bend in the puncture needle based on the first positional information and the second positional information. The processing circuitry is configured to correct a position of information indicating the puncture needle with respect to the ultrasonic image assumed based on the first positional information.

Exemplary embodiments of an ultrasonic diagnostic apparatus are described below in greater detail with reference to the accompanying drawings. In the following description, like components are denoted by like reference numerals, and overlapping explanation thereof is omitted.

### First Embodiment

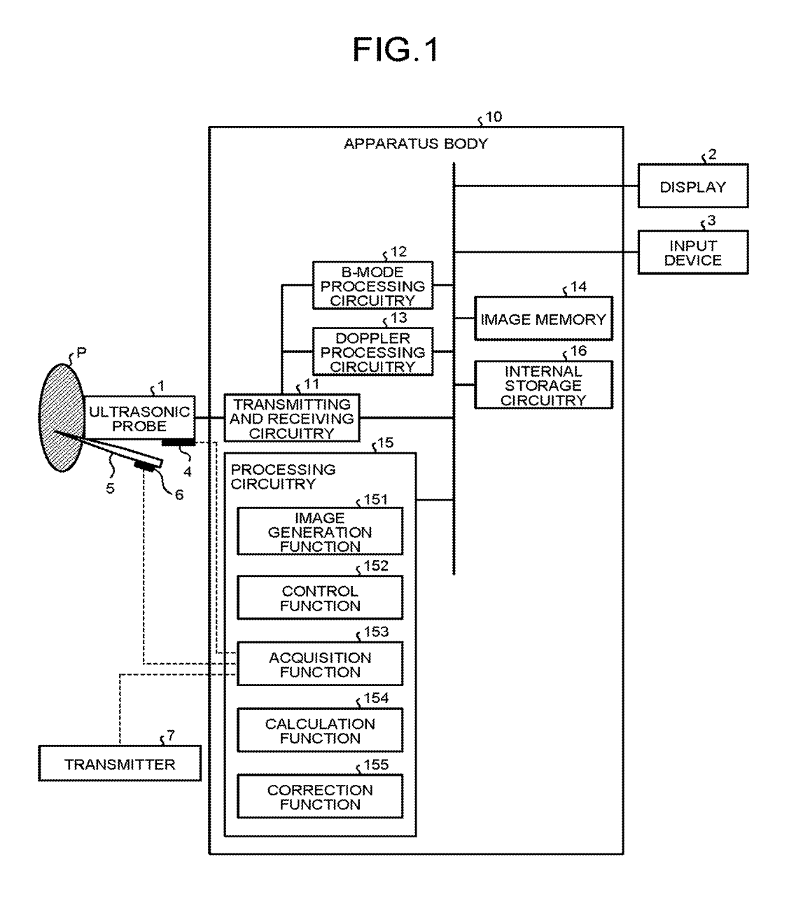

The following describes a configuration of an ultrasonic diagnostic apparatus according to a first embodiment. FIG. 1 is a block diagram of an exemplary configuration of the ultrasonic diagnostic apparatus according to the first embodiment. As illustrated in FIG. 1, the ultrasonic diagnostic apparatus according to the present embodiment includes an ultrasonic probe 1, a display 2, an input device 3, and an apparatus body 10.

The ultrasound probe 1 includes a plurality of piezoelectric transducer elements, for example. The piezoelectric transducer elements generate ultrasound based on driving signals supplied by transmitting and receiving circuitry 11 described below, which is included in the apparatus body 10. The ultrasound probe 1 receive reflected waves from the subject P to convert them into electrical signals. The ultrasound probe 1 further includes a matching layer provided to the piezoelectric transducer elements and a backing member that prevents ultrasound from propagating rearward from the piezoelectric transducer elements. The ultrasound probe 1 is detachably coupled to the apparatus body 10.

When the ultrasound is transmitted from the ultrasound probe 1 to the subject P, the transmitted ultrasound is repeatedly reflected on surfaces of discontinuity of acoustic impedances at tissue in the body of the subject P and is received as reflected-wave signals by the piezoelectric transducer elements of the ultrasound probe 1. The amplitude of the received reflected-wave signal is dependent on the difference between the acoustic impedances on the surface of discontinuity on which the ultrasound is reflected. When a transmitted ultrasound pulse is reflected on the surface of a moving blood flow, a moving cardiac wall, and any other moving object, due to the Doppler effect, the frequency of the reflected-wave signal is shifted depending on the velocity component of the moving object in an ultrasound transmission direction.

The ultrasonic probe 1 according to the first embodiment can scan the subject P two-dimensionally and three-dimensionally with ultrasonic waves. Specifically, the ultrasonic probe 1 according to the first embodiment is a mechanical four-dimensional probe that scans the subject P two-dimensionally with the piezoelectric transducer elements arranged in line and scans the subject P three-dimensionally by oscillating the piezoelectric transducer elements at a predetermined angle (oscillation angle). Alternatively, the ultrasonic probe 1 according to the first embodiment is a two-dimensional probe that can perform ultrasonic scanning three-dimensionally on the subject P with the piezoelectric transducer elements arranged in a matrix. The two-dimensional probe can also scan the subject P two-dimensionally by focusing and transmitting the ultrasonic waves.

A puncture is performed with a puncture needle 5 illustrated in FIG. 1 on a tissue positioned at a region subjected to the ultrasonic scanning by the ultrasonic probe 1 according to the first embodiment. The puncture needle 5 illustrated in FIG. 1, for example, is an electromagnetic needle that generates radio-frequency waves. The puncture needle 5 is connected to a treatment device that controls output of the radio-frequency waves generated by the puncture needle 5. The treatment device can monitor the temperature of the puncture needle 5, output of the radio-frequency waves, and impedance of the cauterization region. A doctor operates the treatment device to perform RFA using the puncture needle 5.

Alternatively, the puncture needle 5 illustrated in FIG. 1, for example, is an electrode needle that applies an electric current to a tissue to be treated. The puncture needle 5 is connected to a treatment device that controls output of the electric current generated by the puncture needle 5. In this case, the treatment device is connected to a plurality of puncture needles 5. The treatment device applies an electric current between the puncture needles, thereby applying the electric current to the tissue to be treated between the puncture needles and performing treatment. The doctor, for example, observes a computed tomography (CT) image or an ultrasonic image acquired in advance to make a treatment plan for a cancer tissue. Examples of the treatment plan include, but are not limited to, how the puncture needles 5 are to be arranged with respect to the cancer tissue, how much electric current is to be applied at how much voltage, etc. The doctor places the puncture needles 5 on the affected area and operates the treatment device while observing the ultrasonic image, thereby performing IRE treatment with the puncture needles 5. The IRE treatment is also called NanoKnife.

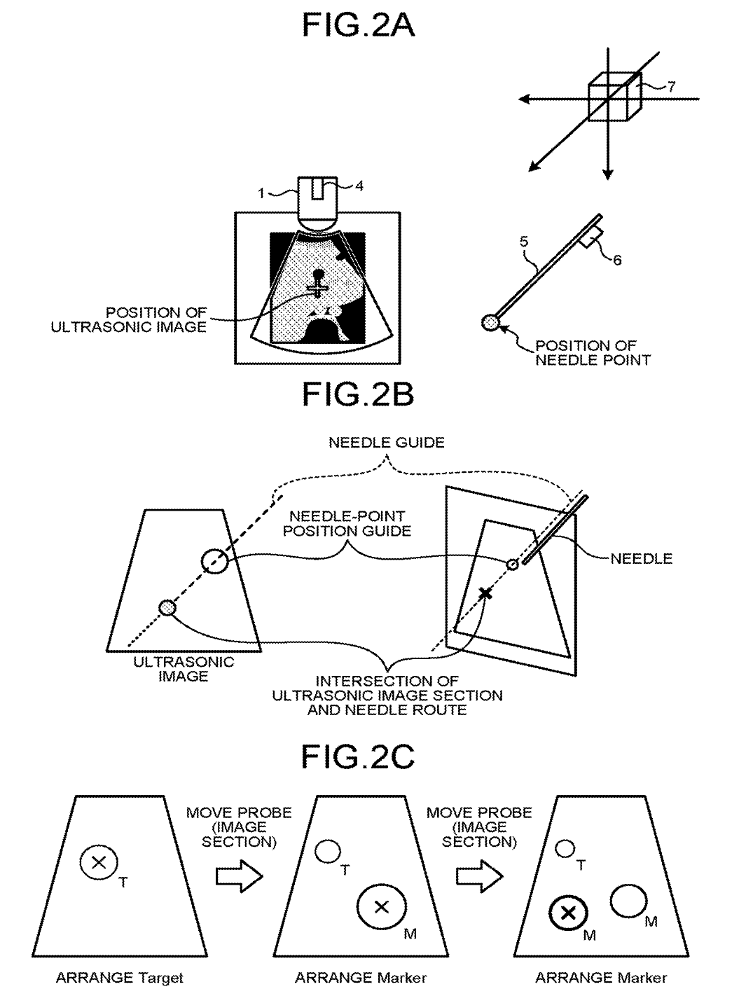

As illustrated in FIG. 1, the ultrasonic probe 1 is provided with a position sensor 4, and the puncture needle 5 is provided with a position sensor 6. A transmitter 7 is arranged at a certain position near the apparatus body 10 according to the first embodiment. The position sensor 4, the position sensor 6, and the transmitter 7 serve as a position detection system that detects positional information on the ultrasonic probe 1 and positional information on the puncture needle 5. FIG. 2A is a diagram for explaining an example of the position detection system according to the first embodiment. The position sensor 4, for example, is a magnetic sensor attached to the ultrasonic probe 1. As illustrated in FIG. 2A, for example, the position sensor 4 is attached to an end of the body of the ultrasonic probe 1. The position sensor 6, for example, is a magnetic sensor attached to the puncture needle 5. As illustrated in FIG. 2A, for example, the position sensor 6 is attached to the proximal end of the puncture needle 5. The transmitter 7, for example, is a device that generates a magnetic field toward the outside of itself.

The position sensor 4 detects the intensity and the inclination of the three-dimensional magnetic field generated by the transmitter 7. Based on the detected information on the magnetic field, the position sensor 4 calculates the position (the coordinates and the angle) of itself in the space with the transmitter 7 as the origin. The position sensor 4 transmits the calculated position to the apparatus body 10. The position sensor 4 transmits the three-dimensional coordinates and angle of itself to the apparatus body 10 as three-dimensional positional information on the ultrasonic probe 1. As a result, the apparatus body 10 can calculate the position of the ultrasonic image in the space with the transmitter 7 as the origin.

The position sensor 6 detects the intensity and the inclination of the three-dimensional magnetic field generated by the transmitter 7. Based on the detected information on the magnetic field, the position sensor 6 calculates the position (the coordinates and the angle) of itself in the space with the transmitter 7 as the origin. The position sensor 6 transmits the calculated position to the apparatus body 10. The position sensor 6 transmits the three-dimensional coordinates and angle of itself to the apparatus body 10 as three-dimensional positional information on the puncture needle 5. Based on the three-dimensional positional information on the puncture needle 5 received from the position sensor 6 (three-dimensional positional information on the position to which the position sensor 6 is attached on the puncture needle 5) and the information on the shape and the size of the puncture needle 5 received in advance, the apparatus body 10 can calculate the position of the needle point of the puncture needle 5 in the space with the transmitter 7 as the origin as illustrated in FIG. 2A.

The present embodiment is also applicable to a case where a system other than the position detection system described above acquires the positional information on the ultrasonic probe 1 and the puncture needle 5. The present embodiment, for example, may be applied to a case where a gyro sensor or an acceleration sensor acquires the positional information on the ultrasonic probe 1 and the puncture needle 5.

As described above, by calculating the position of the ultrasonic image and the position of the puncture needle 5 in the space with the transmitter 7 as the origin, the ultrasonic diagnostic apparatus can calculate the position of the puncture needle 5 with respect to the ultrasonic image. The ultrasonic diagnostic apparatus according to the first embodiment thus can calculate the position of the puncture needle 5 with respect to the ultrasonic image and display a guideline that guides insertion of the puncture needle 5 on the ultrasonic image. FIG. 2B is a diagram of an example of the guideline for the puncture needle according to the first embodiment. The left figure in FIG. 2B illustrates a guideline in an out-of-plane state where the puncture needle is not present in the section of the ultrasonic image. The guideline can also be displayed in an in-plane state where the puncture needle is present in the section of the ultrasonic image (the puncture needle moves forward in the section).

As illustrated in FIG. 2B, for example, the puncture needle guide in the out-of-plane state includes a needle-point position guide and a needle guide. The needle-point position guide indicates the present position of the needle point. The needle guide indicates a route for the needle. Specifically, if the puncture needle at the present position is inserted without any change, the puncture needle moves forward along the needle guide and intersects with the ultrasonic image section at a position represented by an intersection. The doctor, for example, sets in advance a target of treatment (e.g., a cancer tissue) and a marker indicating an organ that should be kept from being punctured with the puncture needle (e.g., a blood vessel) on the ultrasonic image. As a result, the puncture needle guide, the target, and the marker can be displayed on a single screen.

FIG. 2C is a diagram for explaining setting of a target and markers according to the first embodiment. As illustrated in FIG. 2C, for example, the doctor arranges a target “T” while observing the ultrasonic image. Subsequently, the doctor observes the circumference of the target by moving the ultrasonic probe to arrange markers at organs that should be kept from being punctured with the puncture needle. The target and the markers may be arranged on a CT image or the like aligned with the ultrasonic image besides on the ultrasonic image. In treatment using a puncture needle, for example, a CT image or the like is acquired in advance. The doctor determines a target while observing the acquired CT image and makes a treatment plan. Specifically, by aligning the coordinate system of volume data of the CT image with the coordinate system of the space with the transmitter 7 as the origin, the information on the target set on the CT image can be reflected on the ultrasonic image.

Referring back to FIG. 1, the input device 3 includes a mouse, a keyboard, a button, a panel switch, a touch command screen, a foot switch, a trackball, and a joystick, for example. The input device 3 receives various setting requests from an operator of the ultrasonic diagnostic apparatus and transfers the received various setting requests to the apparatus body 10.

The display 2 displays a graphical user interface (GUI) that allows the operator of the ultrasound diagnostic apparatus to input various kinds of setting requests with the input device 3, and displays various image data generated in the apparatus body 10 and any other data.

The apparatus body 10 generates ultrasonic image data based on the reflected wave signals received by the ultrasonic probe 1. The apparatus body 10 according to the first embodiment, for example, can generate two-dimensional ultrasonic image data based on two-dimensional reflected wave data received by the ultrasonic probe 1. Alternatively, the apparatus body 10 according to the first embodiment, for example, can generate three-dimensional ultrasonic image data based on three-dimensional reflected wave data received by the ultrasonic probe 1. The three-dimensional ultrasonic image data is hereinafter referred to as “volume data”.

As illustrated in FIG. 1, the apparatus body 10 includes the transmitting and receiving circuitry 11, B-mode processing circuitry 12, Doppler processing circuitry 13, an image memory 14, processing circuitry 15, and internal storage circuitry 16. The ultrasonic diagnostic apparatus illustrated in FIG. 1 stores processing functions in the internal storage circuitry 16 as computer programs executable by a computer. The transmitting and receiving circuitry 11, the B-mode processing circuitry 12, the Doppler processing circuitry 13, and the processing circuitry 15 serve as processors that read and execute computer programs from the internal storage circuitry 16 to provide functions corresponding to the respective computer programs. In other words, the circuitry that read the respective computer programs have functions corresponding to the read computer programs.

The term “processor” in the description above indicates circuitry, such as a central processing unit (CPU), a graphics processing unit (GPU), an application specific integrated circuit (ASIC), or a programmable logic device (e.g., a simple programmable logic device (SPLD), a complex programmable logic device (CPLD), and a field programmable gate array (FPGA)). The processors read and execute the computer programs stored in the storage circuitry, thereby providing the functions. The computer programs may be directly incorporated in the circuitry of the processor instead of being stored in the storage circuitry. In this case, the processors read and execute the computer programs incorporated in the circuitry, thereby providing the functions. The processors according to the present embodiment are not necessarily provided as respective separate circuits. Alternatively, a plurality of individual circuits may be combined as one processor to provide the respective functions.

The transmitting and receiving circuitry 11 includes a pulse generator, transmission-delaying circuitry, and a pulser, and supplies driving signals to the ultrasound probe 1. The pulse generator repeatedly generates rate pulses for forming transmitted ultrasound at a predetermined rate frequency. Furthermore, the transmission-delaying circuitry gives a delay time for each piezoelectric transducer element to the corresponding rate pulse generated by the pulse generator. Such a delay time is required to converge the ultrasound generated by the ultrasound probe 1 into a beam and determine transmission directionality. Furthermore, the pulser applies the driving signals (driving pulses) to the ultrasound probe 1 at a timing based on the rate pulses. That is, the transmission-delaying circuitry desirably adjusts the transmission direction of the ultrasound transmitted from the surface of the piezoelectric transducer elements, by varying the delay time given to each rate pulse.

The transmitting and receiving circuitry 11 has a function to be able to instantly change, for example, a transmission frequency and a transmission driving voltage, to perform a predetermined scanning sequence based on instructions from the processing circuitry 15 as described below. In particular, the change in the transmission driving voltage is achieved by linear-amplifier-type oscillation circuitry that is capable of instantly switching the value of the voltage, or by a mechanism that electrically switches a plurality of power sources.

The transmitting and receiving circuitry 11 further includes a preamplifier, an analog/digital (A/D) converter, reception delay circuitry, and an adder, for example. The transmitting and receiving circuitry 11 performs various types of processing on the reflected wave signals received by the ultrasonic probe 1, thereby generating reflected wave data. The preamplifier amplifies the reflected wave signals in each channel. The A/D converter performs A/D conversion on the amplified reflected wave signals. The reception delay circuitry supplies a delay time required to determine the reception directivity. The adder performs addition on the reflected wave signals processed by the reception delay circuitry, thereby generating reflected wave data. The addition performed by the adder emphasizes a reflection component in a direction corresponding to the reception directivity of the reflected wave signals. Based on the reception directivity and the transmission directivity, a synthetic beam for transmitting and receiving ultrasonic waves is formed.

To scan the subject P two-dimensionally, the transmitting and receiving circuitry 11 according to the first embodiment causes the ultrasonic probe 1 to transmit a two-dimensional ultrasonic beam. The transmitting and receiving circuitry 11 according to the first embodiment generates two-dimensional reflected wave data from two-dimensional reflected wave signals received by the ultrasonic probe 1. To scan the subject P three-dimensionally, the transmitting and receiving circuitry 11 according to the first embodiment causes the ultrasonic probe 1 to transmit a three-dimensional ultrasonic beam. The transmitting and receiving circuitry 11 according to the first embodiment generates three-dimensional reflected wave data from three-dimensional reflected wave signals received by the ultrasonic probe 1.

Various forms may be selected as the form of output signals from the transmitting and receiving circuitry 11, including signals containing phase information, which are called radio-frequency (RF) signals, and amplitude information resulting from envelope detection, for example.

The B-mode processing circuitry 12 receives reflected wave data from the transmitting and receiving circuitry 11. The B-mode processing circuitry 12 performs logarithmic amplification, envelope detection, and other processing on the reflected wave data, thereby generating data (B-mode data) indicating the signal intensity as the intensity of luminance. The Doppler processing circuitry 13 performs a frequency analysis on velocity information obtained from reflected wave data received from the transmitting and receiving circuitry 11. The Doppler processing circuitry 13 extracts a bloodstream, a tissue, and a contrast medium echo component by the Doppler effect and generates data (Doppler data) by extracting moving object information, such as velocity, dispersion, and power, at multiple points. The moving object according to the present embodiment is a fluid, such as blood flowing in blood vessels and lymph flowing in lymphatic vessels.

The B-mode processing circuitry 12 and the Doppler processing circuitry 13 according to the first embodiment can process both two-dimensional reflected wave data and three-dimensional reflected wave data. Specifically, the B-mode processing circuitry 12 generates two-dimensional B-mode data from the two-dimensional reflected wave data and generates three-dimensional B-mode data from the three-dimensional reflected wave data. The Doppler processing circuitry 13 generates two-dimensional Doppler data from the two-dimensional reflected wave data and generates three-dimensional Doppler data from the three-dimensional reflected wave data. In the three-dimensional B-mode data, the luminance values corresponding to the reflection intensities of reflection sources are allocated to respective points (sample points) set on scanning lines in the range of three-dimensional scanning. In the three-dimensional Doppler data, the luminance values corresponding to the values of bloodstream information (velocity, dispersion, and power) are allocated to respective points (sample points) set on the scanning lines in the range of three-dimensional scanning.

The image memory 14 stores therein image data for display generated by the processing circuitry 15, which will be described later. The image memory 14 can also store therein data generated by the B-mode processing circuitry 12 and the Doppler processing circuitry 13. The B-mode data and the Doppler data stored in the image memory 14 can be retrieved by the operator after a diagnosis, for example. The B-mode data and the Doppler data are converted into ultrasonic image data for display via the processing circuitry 15.

The internal storage circuitry 16 stores therein a control program for performing transmission and reception of ultrasonic waves, image processing, and display processing, and various types of data, such as diagnosis information (e.g., a patient ID and findings of the doctor), a diagnosis protocol, and various body marks. The internal storage circuitry 16, for example, is also used to hold image data stored in the image memory 14 as needed. The data stored in the internal storage circuitry 16 may be transferred to an external device via an interface, which is not illustrated.

The processing circuitry 15 collectively controls the processing of the ultrasonic diagnostic apparatus. Specifically, the processing circuitry 15 reads and executes, from the internal storage circuitry 16, computer programs corresponding to an image generation function 151, a control function 152, an acquisition function 153, a calculation function 154, and a correction function 155 illustrated in FIG. 1, thereby performing various types of processing. The processing circuitry 15, for example, controls the processing of the transmitting and receiving circuitry 11, the B-mode processing circuitry 12, and the Doppler processing circuitry 13 based on various setting requests received from the operator via the input device 3 and various control programs and various types of data read from the internal storage circuitry 16. The processing circuitry 15 performs control so as to display, on the display 2, the ultrasonic image data for display stored in the image memory 14 and the internal storage circuitry 16. The processing circuitry 15 also performs control so as to display processing results on the display 2. The processing circuitry 15, for example, reads and executes the computer program corresponding to the control function 152, thereby collectively controlling the apparatus and performing the processing described above.

The image generation function 151 generates ultrasonic image data from the data generated by the B-mode processing circuitry 12 and the Doppler processing circuitry 13. Specifically, the image generation function 151 generates B-mode image data indicating the intensities of the reflected waves as the luminance from the two-dimensional B-mode data generated by the B-mode processing circuitry 12. The B-mode image data corresponds to data obtained by extracting a tissue shape in the region subjected to ultrasonic scanning. The image generation function 151 also generates Doppler image data indicating the moving object information from the two-dimensional Doppler data generated by the Doppler processing circuitry 13. The Doppler image data is velocity image data, dispersion image data, power image data, or image data obtained by combining these image data. The Doppler image data corresponds to data indicating fluid information on a fluid flowing in the region subjected to ultrasonic scanning.

The image generating function 151 typically converts (performs scan conversion) a scanning-line signal sequence from an ultrasound scan into a scanning-line signal sequence in a video format typified by, for example, television and generates ultrasound image data for display. Specifically, the image generating function 151 generates the ultrasound image data for display by performing coordinate transformation according to an ultrasound scanning mode used by the ultrasound probe 1. Furthermore, in addition to the scan conversion, the image generating function 151 performs various types of image processing, for example, using a plurality of image frames after the scan conversion. Examples of such image processing include image processing (smoothing processing) that regenerates an average image of brightness, and image processing (edge enhancement processing) that uses a differential filter within an image. In addition, the image generating function 151 combines the ultrasound image data with text information on various parameters, scales, and body marks, for example.

That is, the B-mode data and the Doppler data are ultrasound image data before the scan conversion processing, whereas data generated by the image generating function 151 is ultrasound image data for display after the scan conversion processing. The B-mode data and the Doppler data are also referred to as “raw data”.

The image generation function 151 performs coordinate conversion on the three-dimensional B-mode data generated by the B-mode processing circuitry 12, thereby generating three-dimensional B-mode image data. The image generation function 151 also performs coordinate conversion on the three-dimensional Doppler data generated by the Doppler processing circuitry 13, thereby generating three-dimensional Doppler image data. The three-dimensional B-mode data and the three-dimensional Doppler data correspond to volume data yet to be subjected to scan-conversion. In other words, the image generation function 151 generates “the three-dimensional B-mode image data and the three-dimensional Doppler image data” as “volume data serving as three-dimensional ultrasonic image data”.

To generate various types of two-dimensional image data for displaying volume data on the display 2, the image generation function 151 performs rendering on the volume data. Examples of the rendering performed by the image generation function 151 include, but are not limited to: performing multi-planer reconstruction (MPR) to generate MPR image data from volume data, performing “curved MPR” on the volume data, performing “maximum intensity projection” on the volume data, volume rendering (VR) for generating two-dimensional image data reflecting three-dimensional information, etc.

The image generation function 151 can perform the various types of rendering described above on volume data acquired by other medical image diagnostic apparatuses. The volume data corresponds to three-dimensional X-ray CT image data (X-ray CT volume data) acquired by an X-ray CT apparatus or three-dimensional magnetic resonance imaging (MRI) image data (MRI volume data) acquired by an MRI apparatus. The image generation function 151, for example, performs MPR on a section corresponding to the scanning section of the two-dimensional ultrasonic image generated at this time based on the positional information on the ultrasonic probe 1 acquired by the acquisition function 153. The image generation function 151 thus reconstructs MPR image data of the section image from the volume data.

The control function 152 performs various types of control described above on the whole apparatus. The acquisition function 153 acquires information on the position of the puncture needle 5. The calculation function 154 calculates information on a bend in the puncture needle 5. The correction function 155 corrects a bend in the puncture needle 5. These functions will be described later in greater detail.

The explanation has been made of the entire configuration of the ultrasonic diagnostic apparatus according to the first embodiment. When a procedure using the puncture needle 5 is performed, for example, the ultrasonic diagnostic apparatus according to the first embodiment having the configuration described above improves the workflow of the procedure. In a procedure using the puncture needle, a doctor moves the puncture needle to a target while observing the puncture needle guide to perform RFA or IRE. The puncture needle guide is displayed based on the positional information acquired by the position sensor attached to the puncture needle. Specifically, the position sensor is attached to the proximal end of the currently used puncture needle to calculate the position of the distal end of the puncture needle based on the shape and the size of the puncture needle. The puncture needle guide thus displays an extension of the line segment between the proximal end and the distal end as the needle guide.

In a procedure using the puncture needle, however, the puncture needle may possibly be bent by a hard tissue or the weight of the position sensor and a cable. As a result, the needle guide is displayed in a manner deviating from the actual position of the needle. When the needle guide is displayed in a manner deviating from the actual position, the puncture needle is inserted into a position different from the position indicated by the puncture needle guide even if it is inserted along the needle guide. If the puncture needle is inserted near the target, the target is not present there. As a result, the puncture needle needs to be reinserted, thereby deteriorating the efficiency of the procedure. To address this, the ultrasonic diagnostic apparatus according to the first embodiment calculates a bend in the puncture needle based on the position of the puncture needle and corrects the puncture needle guide based on the calculated bend, thereby improving the workflow of the procedure. The processing performed by the ultrasonic diagnostic apparatus according to the first embodiment will be described in greater detail. The following describes processing performed after a series of alignment is performed using the position sensor 4 and the position sensor 6. Specifically, alignment is performed in advance so as to acquire the positions of the subject, the ultrasonic probe 1, and the puncture needle 5 in a space from which an ultrasonic image is acquired (coordinate space formed by the transmitter 7).

The acquisition function 153 illustrated in FIG. 1 acquires first positional information and second positional information. The first positional information indicates the position of the puncture needle 5 in the space from which the ultrasonic image is acquired. The second positional information indicates the position of the puncture needle 5 included in the ultrasonic image. Specifically, the acquisition function 153 acquires the positional information on the puncture needle 5 in the space from which the ultrasonic image is acquired and the position of the puncture needle 5 displayed in the ultrasonic image. The acquisition function 153, for example, acquires the positional information on the puncture needle 5 in the space from which the ultrasonic image is acquired based on the information transmitted from the position sensor 4 attached to the ultrasonic probe 1 and the position sensor 6 attached to the puncture needle 5.

The acquisition function 153 also acquires the position of the puncture needle 5 actually displayed on the ultrasonic image and specified by the operator. When the mode is shifted to a needle guide correction mode, for example, the control function 152 displays, on the display 2, a screen that instructs the operator to specify the position of the puncture needle 5 on the ultrasonic image. In response to this, the operator moves the ultrasonic probe 1 such that the puncture needle 5 is displayed on the ultrasonic image and specifies the position of the puncture needle 5 displayed on the ultrasonic image with the input device 3. The acquisition function 153 acquires the positional information specified by the operator.

The positional information on the puncture needle 5 displayed on the ultrasonic image may be automatically extracted instead of being specified by the operator. In this case, the acquisition function 153, for example, extracts a high-luminance area in the ultrasonic image as the position of the puncture needle 5. Alternatively, after the extraction is automatically performed, the operator may select the positional information. The acquisition function 153, for example, may extract a plurality of high-luminance areas from the ultrasonic image and allow the operator to select an area from the extracted areas.

The calculation function 154 illustrated in FIG. 1 calculates a bend in the puncture needle based on the first positional information and the second positional information. Specifically, the calculation function 154 calculates the degree of a bend in the puncture needle 5 based on the position of the puncture needle 5 in the space from which the ultrasonic image is acquired and the position of the puncture needle 5 in the ultrasonic image acquired by the acquisition function 153. The calculation function 154 uses information on the puncture needle guide besides the positional information described above. Specifically, the calculation function 154 calculates a bend in the puncture needle 5 using the guideline for the puncture needle 5 set based on the position of the puncture needle 5 in the space from which the ultrasonic image is acquired and using the position of the puncture needle in the actual ultrasonic image.

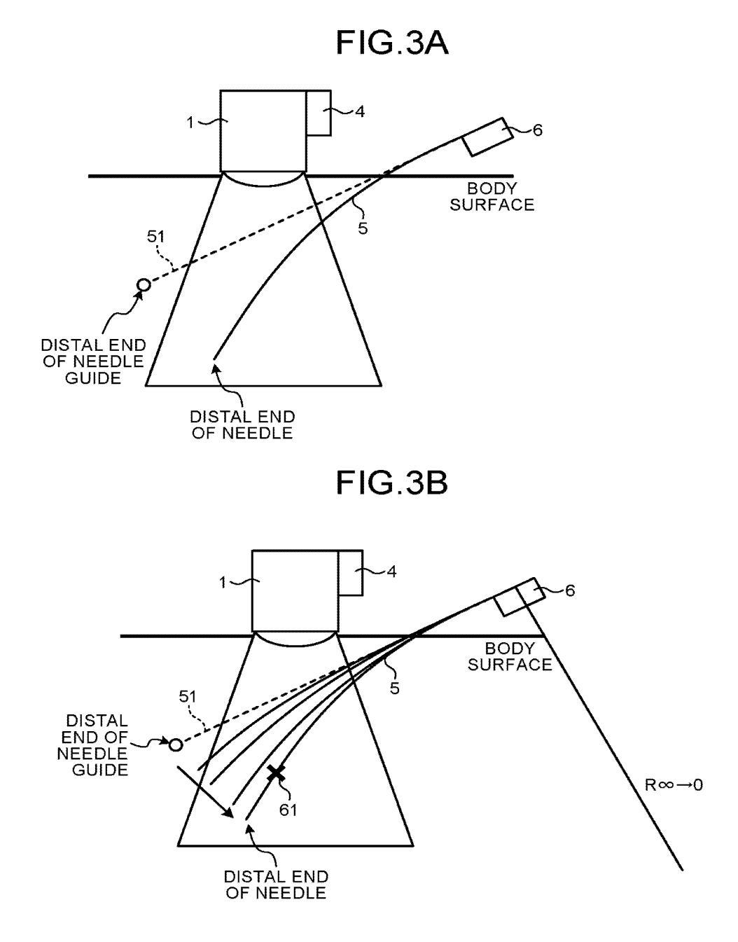

FIGS. 3A and 3B are diagrams for explaining an example of calculation of a bend in the puncture needle 5 according to the first embodiment. If the puncture needle 5 inserted from a body surface is bent by insertion into a hard tissue or a cable connected to the puncture needle 5, for example, a guideline 51 deviates from the actual puncture needle 5 as illustrated in FIG. 3A. This is because the distal end of the needle guide is calculated based on the positional information on the position sensor 6 and the shape and the size of the puncture needle. If the puncture needle 5 is bent, the information on the bend in not acquired, resulting in deviation of the guideline from the puncture needle.

If the positional information on the puncture needle 5 in the ultrasonic image is acquired in the state illustrated in FIG. 3A, the calculation function 154 calculates a bend in the puncture needle 5 using the guideline, the position of the puncture needle 5 in the ultrasonic image, and the positional information on the position sensor 6. As illustrated in FIG. 3B, for example, the calculation function 154 calculates a bend in the puncture needle 5 by superposing the guideline 51 on a curve passing through a position 61 of the puncture needle 5 in the ultrasonic image and the position of the position sensor 6. For example, the calculation function 154 changes the radius of curvature of a circle passing through the position sensor 6 from infinity to zero in the plane including the guideline 51 and the position 61. The calculation function 154 derives the curvature at which the circle intersects with the position 61 as the bend in the puncture needle 5. Specifically, as illustrated in FIG. 3B, the calculation function 154 gradually decreases the radius from that of a circle having an infinite radius of curvature, searches for a circle passing through the position sensor 6 and the position 61, and derives the curvature of the retrieved circle as the bend in the puncture needle 5.

The calculation method illustrated in FIGS. 3A and 3B is given by way of example only, and the calculation function 154 may calculate a bend in the puncture needle 5 by another calculation method. The calculation function 154, for example, may calculate a bend in the puncture needle 5 by acquiring not one point but a plurality of points of positions on the actual ultrasonic image and performing elliptic or Bezier interpolation on the acquired points. Because a bend caused by the hardness of a tissue and a bend caused by the weight of the sensor and the cable do not correspond to a circle, for example, the calculation method using a plurality of points described above is likely to calculate the bend with high accuracy.

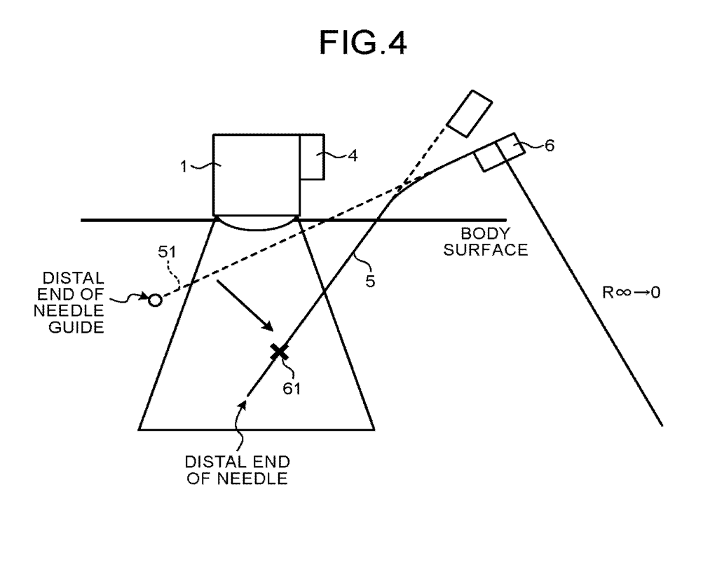

While the calculation method illustrated in FIGS. 3A and 3B calculates a bend assuming that the puncture needle 5 is bent as a whole, the calculation function 154 may calculate a bend in the puncture needle 5 assuming that the puncture needle 5 keeps its straight-line shape in the body. FIG. 4 is a diagram for explaining another example of calculation of a bend in the puncture needle according to the first embodiment. FIG. 4 illustrates an example where only a portion of the puncture needle 5 outside the body surface is bent. In this case, as illustrated in FIG. 4, the calculation function 154 changes the radius of curvature of a circle passing through the position sensor 6 from infinity to zero in the plane including a portion of the guideline outside the body surface and the position 61 with a portion of the guideline 51 inside the body keeping its straight-line shape. The calculation function 154 derives the curvature at which the circle intersects with the position 61 as the bend in the puncture needle 5.

Referring back to FIG. 1, the correction function 155 corrects the position of information indicating the puncture needle with respect to the ultrasonic image assumed based on the first positional information. Specifically, the correction function 155 corrects the guideline for the puncture needle 5 based on the bend in the puncture needle 5 calculated by the calculation function 154. The correction function 155, for example, displays the guideline for the puncture needle fixed based on the bend in the curvature calculated by the calculation function 154. In other words, the correction function 155 corrects the guideline already derived and displayed with the bend in the curve calculated by the calculation function 154.

As described above, the ultrasonic diagnostic apparatus according to the first embodiment can calculate a bend in the puncture needle 5 and correct the guideline for the puncture needle. This configuration enables the operator to perform a procedure while referring to the corrected guideline, thereby improving the workflow of the procedure. The following describes an example of display information with the corrected guideline.

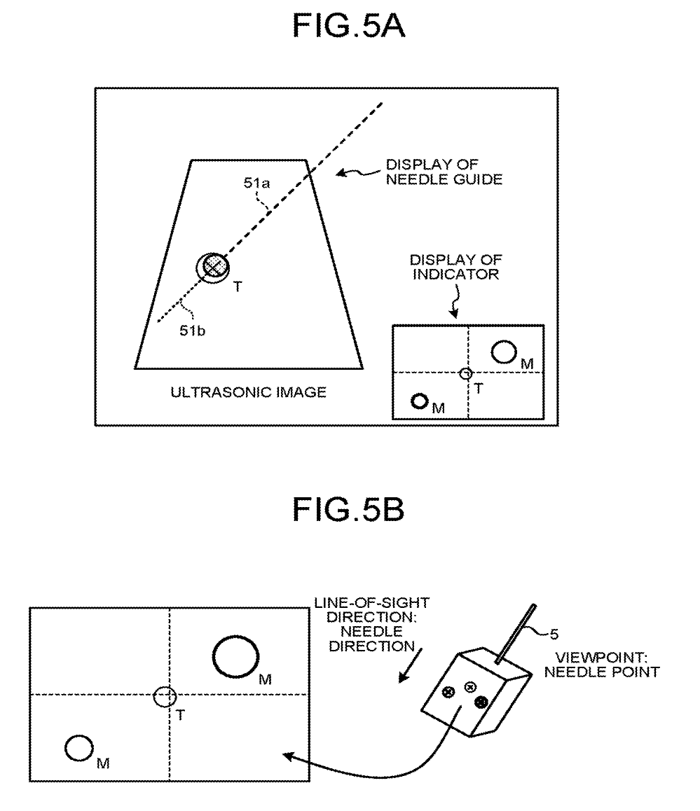

The control function 152 illustrated in FIG. 1 displays, on the display 2, information based on the position of information indicating the puncture needle corrected by the correction function 155, for example. The following describes examples of display information with reference to FIGS. 5A, 5B, 6A, 6B, 7, 8, 9, 10A, 10B, 11A, and 11B. FIGS. 5A to 11B are diagrams of examples of display information according to the first embodiment. As illustrated in FIG. 5A, for example, the control function 152 displays a corrected needle guide on the ultrasonic image. As illustrated in FIG. 5A, the control function 152 displays the needle guide such that a portion of the needle guide before an intersection with the ultrasonic image has a different color from that of a portion after the intersection. The control function 152 thus displays an image that facilitates the operator's understanding of the arrangement state of the needle guide with respect to the ultrasonic image. In a case where the target “T” is displayed on the ultrasonic image as illustrated in FIG. 5A, for example, the operator operates the puncture needle 5 such that the target “T” is positioned at the intersection of the needle guide and the ultrasonic image.

As illustrated in FIG. 5A, the control function 152 may display an indicator together with the ultrasonic image. The indicator, for example, is displayed so as to indicate the positions of the target and the markers with the needle point defined as a viewpoint. To facilitate the operator's understanding to which side the puncture needle 5 needs to be inclined with respect to the probe, for example, the control function 152 generates and displays the indicator assuming the projection area is displayed based on the probe. For example, the control function 152 defines “TOP (upper side)” and “RIGHT (right side)” in the indicator based on a probe section (ultrasonic image). The control function 152, for example, defines the far side with respect to the probe section as “TOP” in the indicator and defines the right side with respect to the probe section as “RIGHT” in the indicator.

To create the indicator relating to the puncture needle 5 in the three-dimensional space illustrated in FIG. 5B, for example, the control function 152 sets the direction of movement of the needle as a line-of-sight direction with the needle point of the puncture needle defined as the viewpoint. In other words, the control function 152 displays the indicator that seems to move forward as the puncture needle 5 is inserted and moved forward. The control function 152 displays the indicator such that the side closer to the ultrasonic image than the puncture needle 5 (far side in the section of the ultrasonic image) corresponds to the upper side in the indicator and that the right side with respect to the ultrasonic image corresponds to the right side in the indicator.

In a case where the indicator is created by performing parallel projection on the three-dimensional space illustrated in FIG. 5B, the size of the target and the markers in the three-dimensional space remains the same. By contrast, in a case where the indicator is created by performing perspective projection, the size of the target and the markers in the three-dimensional space changes depending on the distance from the needle point. If the distance to the target is long, for example, the control function 152 displays the indicator obtained by parallel projection. By contrast, if the distance to the target is short, the control function 152 displays the indicator obtained by perspective projection. With this configuration, the operator does not lose the direction of the target when the distance to the target is short and instinctively grasps the distance to the target when the needle point comes closer to the target. By operating the puncture needle 5 such that the target is positioned at the center of the indicator, for example, the operator can accurately insert the puncture needle 5 into the target.

The control function 152, for example, may also display a body mark or an image of an actual organ in the indicator. If the control function 152 displays only the target and the markers in the indicator, for example, the operator fails to find out through which part in the actual body the puncture needle 5 is passing. Displaying an organ or the like in association with movement of the puncture needle enables the operator to grasp the positional relation of the puncture needle 5 in the body. The control function 152 may also display the corrected guideline in the indicator.

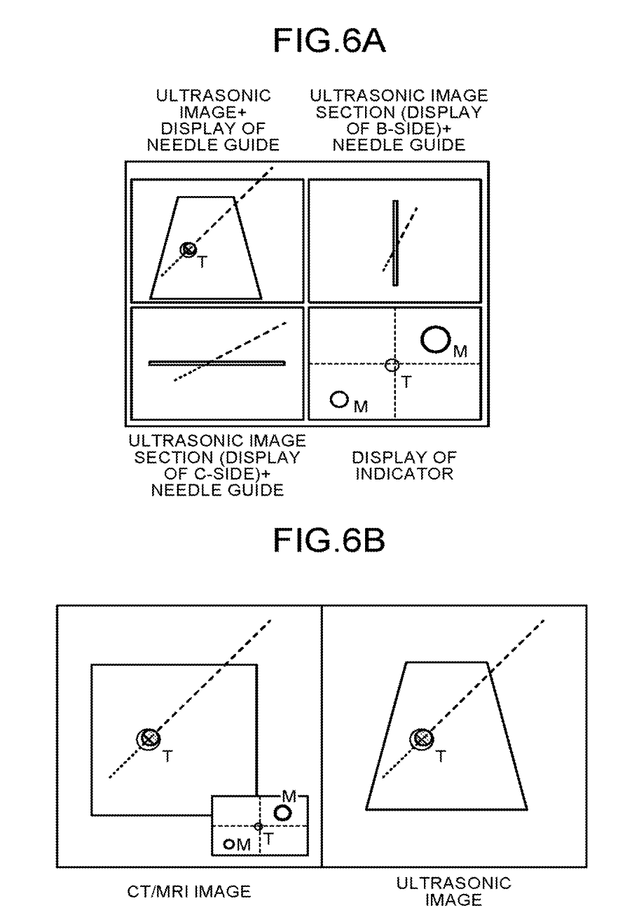

The control function 152 may display ultrasonic images of orthogonal three sections and the indicator on the display 2. As illustrated in FIG. 6A, for example, the control function 152 may display the ultrasonic images of orthogonal three sections with the corrected needle guide displayed thereon together with the indicator. Displaying these pieces of display information can facilitate the operator's inserting the puncture needle 5, thereby improving the workflow of the procedure.

The control function 152 may display the corrected needle guide on an image of another modality. As illustrated in FIG. 6B, for example, the control function 152 may display the ultrasonic image with the corrected needle guide displayed thereon together with a CT image or an MRI image, which is aligned with the ultrasonic image, with the corrected needle guide displayed thereon.

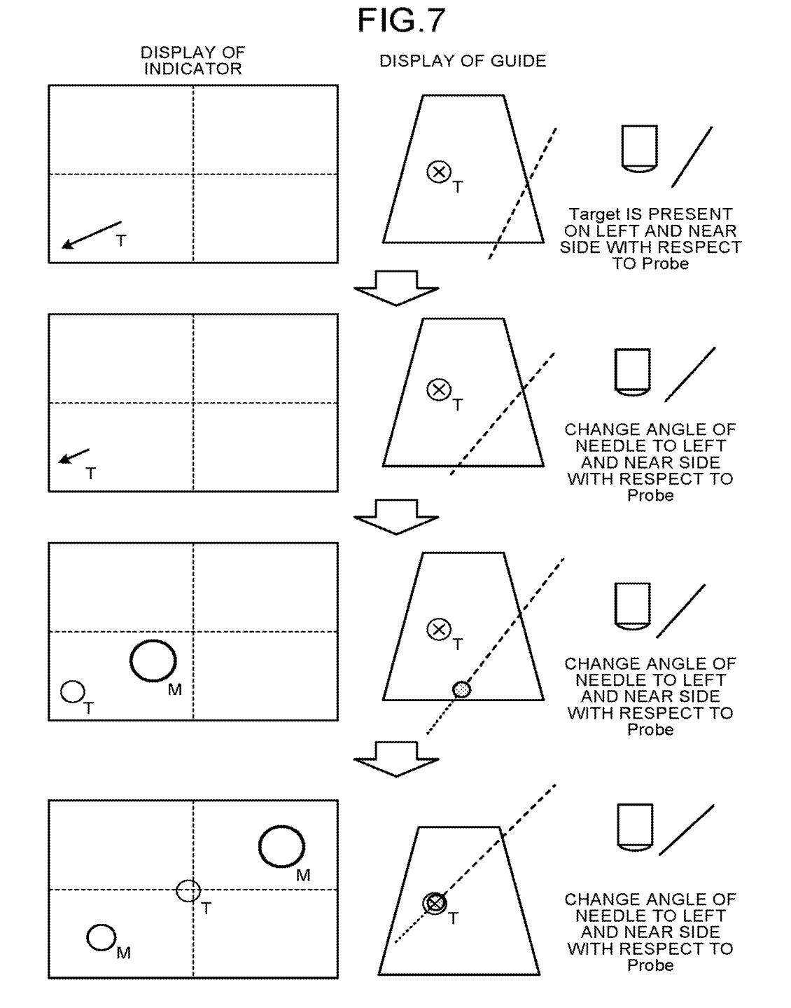

The control function 152 may support insertion of the puncture needle 5 using the indicator. As illustrated in the top figure in FIG. 7, for example, the control function 152 displays an arrow indicating the direction of the target in the indicator displayed together with the needle guide. Because the arrow in the indicator points lower left, it is found that the target is present on the left and the near side with respect to the probe section. The operator operates the puncture needle 5 such that its distal end faces the left and the near side with respect to the probe section, thereby turning it to the target.

When the distance to the target becomes shorter, the control function 152 shortens the arrow indicating the direction of the target as illustrated in the second figure from the top in FIG. 7. The operator further operates puncture needle 5 such that its distal end faces the left and the near side with respect to the probe section while viewing the indicator. As a result, the operator can bring the target into the indicator as illustrated in the third and the fourth figures from the top in FIG. 7. By operating the puncture needle 5 such that the target is positioned at the center of the indicator, the operator can turn the puncture needle 5 to the target while observing the ultrasonic image.

The control function 152 may optionally determine the definition of “TOP” and “RIGHT” in the indicator with respect to the probe section. The control function 152, for example, defines “TOP” as the far side with respect to the probe section and “RIGHT” as the left side with respect to the probe section. The target and the markers in the indicator do not necessarily have a circular shape and may be a three-dimensional area subjected to Bezier interpolation or the like based on information traced from a plurality of sections.

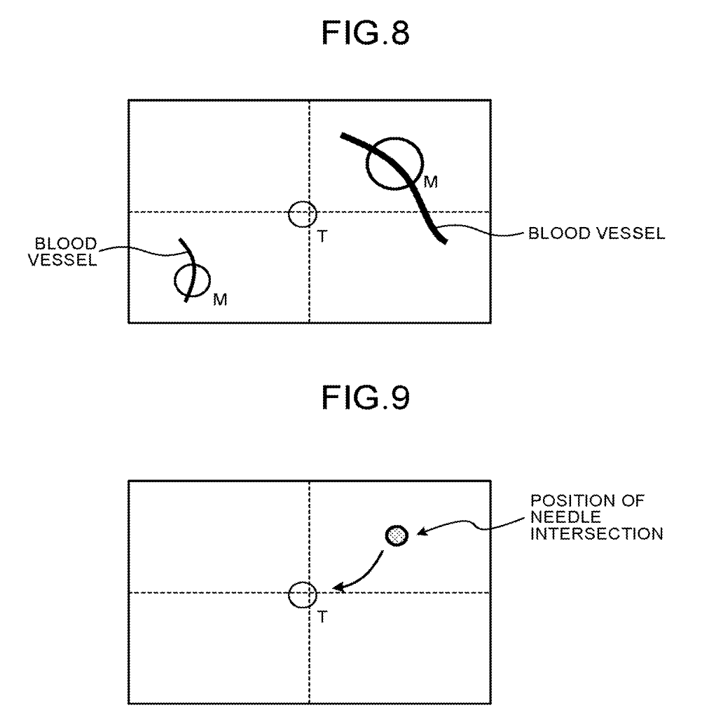

The control function 152 may display the target and the markers with blood vessel information acquired by the color Doppler technique projected thereto. As illustrated in FIG. 8, for example, the control function 152 may display an indicator with blood vessels corresponding to the respective markers in the indicator projected thereto.

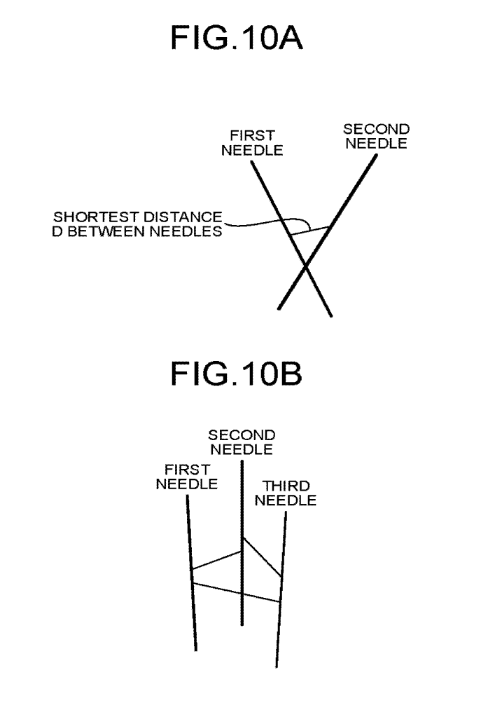

The control function 152 may display a second indicator with the target fixed to the center thereof besides the indicator with the needle point fixed to the center thereof. In the example above, changing the direction of the puncture needle 5 causes the target to get into the indicator, whereas the needle point gets into the second indicator. As illustrated in FIG. 9, for example, the control function 152 displays the second indicator with the target fixed to the center thereof. As illustrated in FIG. 9, the operator operates the puncture needle such that the point indicating the intersection of the puncture needle 5 is positioned at the center of the indicator.

The control function 152 may display a three-dimensional indicator besides the two-dimensional indicator described above. In a case where a multi-needle (e.g., Celon) is used for local treatment for a liver cancer, for example, RFA is performed with the tumor sandwiched between a plurality of puncture needles. It is difficult, however, to accurately grasp the positional relation between the puncture needles two-dimensionally. To address this, the control function 152 may display the positional relation between the puncture needles and the target in the indicator.

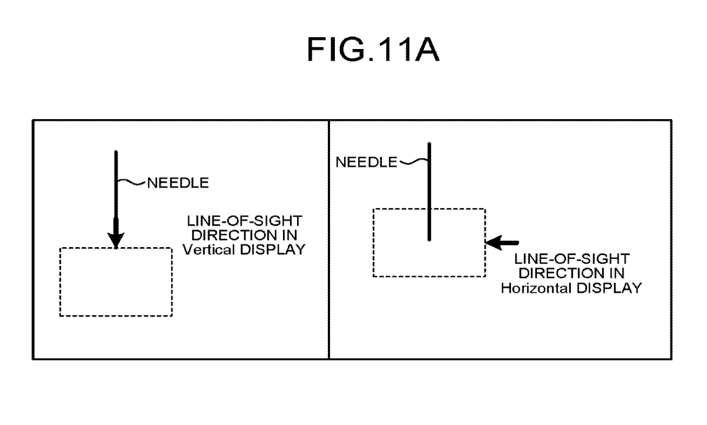

As illustrated in FIG. 10A, for example, the control function 152 displays an indicator indicating the positional relation between a first needle and a second needle three-dimensionally. The three-dimensional indicator is generated in a manner capable of being rotated so that the operator can check the positional relation between the puncture needles from any desired direction. Specifically, the operator performs an operation with the input device 3 to rotate the three-dimensional indicator illustrated in FIG. 10A in a desired direction, for example. As a result, the operator can observe the positional relation between the first needle and the second needle in a desired direction. The control function 152 may also display a shortest distance “D” between the needles in the indicator. In treatment using a plurality of puncture needles, for example, the puncture needles 5 may be arranged not only in parallel but also helically. The helical arrangement of the puncture needles enables more effective ablation in some procedures. In this case, the control function 152 displays the distance “D” between the needles in the indicator as a reference of the cauterization region. Alternatively, the control function 152 may display a sphere having a diameter of the distance “D” between the puncture needles. The distance between the needles is calculated based on the distance between the coordinates of the needle guides (or the actual needles).

In a case where the number of the puncture needles is three or more as illustrated in FIG. 10B, for example, the control function 152 may extract line segments corresponding to respective shortest distances between the puncture needles and display a sphere having its center derived from the centers of gravity of three spheres with the respective line segments as the diameter. Also in the example illustrated in FIG. 10B, the operator can rotate the three-dimensional indicator in a desired direction to observe the positional relation between the puncture needles in a desired direction.

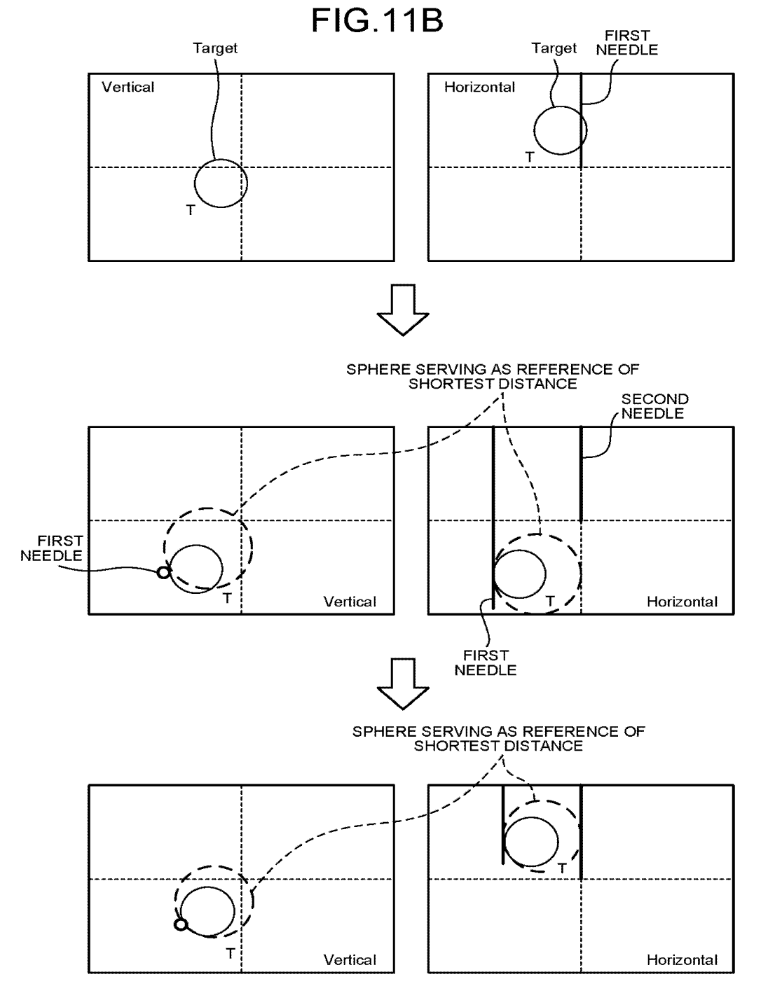

While the direction of movement of the puncture needle 5 corresponds to the line-of-sight direction in the indicator in the example above, the line-of-sight direction in the indicator may be set to any desired direction. The control function 152 may display an indicator indicating the puncture needle 5 viewed from the side, for example. In this case, the control function 152 may perform “vertical display” and “horizontal display” as illustrated in FIG. 11A, for example. In the “vertical display”, the direction of movement of the puncture needle 5 (longitudinal direction of the puncture needle 5) corresponds to the line-of-sight direction in the indicator. In the “horizontal display”, a direction (horizontal direction) orthogonal to the longitudinal direction of the puncture needle 5 corresponds to the line-of-sight direction in the indicator.

The following describes exemplary use of the indicator that performs the “vertical display” and the “horizontal display” with reference to FIG. 11B. FIG. 11B illustrates arrangement of the puncture needles in treatment performed with the target sandwiched between two puncture needles 5. As illustrated in FIG. 11B, for example, the control function 152 displays the indicator including the “vertical display” and the “horizontal display”. As illustrated in the top figure in FIG. 11B, for example, the operator inserts the first puncture needle such that the target comes into contact with the center of the indicator in the “vertical display” and that the first puncture needle exceeds the lower end of the target (that the cauterization portion of the puncture needle is appropriately arranged with respect to the target) in the “horizontal display”.

When the operator starts to insert the second puncture needle, the control function 152 displays a sphere with the shortest distance between the puncture needles as the diameter (sphere indicating a region an internal tissue of which is to be cauterized) in the indicator as illustrated in the middle figure in FIG. 11B. Specifically, the operator inserts the second puncture needle such that the target is included in the sphere. In a case where the second puncture needle is arranged as illustrated in the middle figure in FIG. 11B, for example, the operator can find out that the target is included in the sphere in the “horizontal display”, but the whole target is not included in the sphere in the “vertical display”.

The operator operates again (reinserts) the second puncture needle to find out that the target is included in the sphere both in the “vertical display” and in the “horizontal display” as illustrated in the bottom figure in FIG. 11B. As a result, the operator can reliably cauterize the whole target. In these display, the control function 152 indicates the sphere serving as the reference of the shortest distance with a dotted line until the position of the puncture needles is determined as illustrated in FIG. 11B.

As described above, the control function 152 can display various indicators. Furthermore, the control function 152 can make notification of re-correction. If the bend rate of a puncture needle already arranged (inserted and fixed with a lock tool) changes in a procedure using a plurality of puncture needles, for example, the control function 152 performs control to carry out correction again. If the corrected positional relation between the position sensors of two or more puncture needles changes from that obtained at the time of correction, for example, the control function 152 makes notification of re-correction.

If a puncture needle to be corrected is detected, for example, the control function 152 changes the color of the guideline for the puncture needle, displays the guideline in a blinking manner, or changes display of a precision value (confidence value). Alternatively, if a change in the positional information is equal to or larger than a predetermined threshold, the control function 152 cancels the previous correction of the puncture needle and makes notification of re-correction thereof.

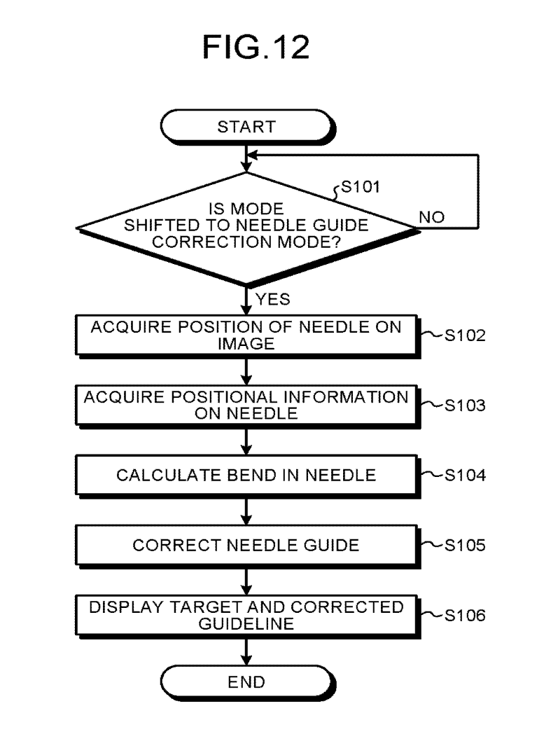

The following describes processing performed by the ultrasonic diagnostic apparatus according to the first embodiment. FIG. 12 is a flowchart for explaining exemplary processing performed by the ultrasonic diagnostic apparatus according to the first embodiment. Processing at Step S101 in FIG. 12 is performed by the processing circuitry 15 reading and executing the computer program corresponding to the control function 152 from the internal storage circuitry 16. At Step S101, the processing circuitry 15 determines whether the mode is shifted to the needle guide correction mode. Processing at Step S102 and processing at S103 are performed by the processing circuitry 15 reading and executing the computer program corresponding to the acquisition function 153 from the internal storage circuitry 16. If it is determined that the mode is the needle guide correction mode (Yes at Step S101), the processing circuitry 15 acquires the position of the needle on the image at Step S102. At Step S103, the processing circuitry 15 acquires the positional information on the needle.

Processing at Step S104 in FIG. 12 is performed by the processing circuitry 15 reading and executing the computer program corresponding to the calculation function 154 from the internal storage circuitry 16. At Step S104, the processing circuitry 15 calculates a bend in the needle. Processing at Step S105 in FIG. 12 is performed by the processing circuitry 15 reading and executing the computer program corresponding to the correction function 155 from the internal storage circuitry 16. At Step S105, the processing circuitry 15 corrects the needle guide.

Processing at Step S106 in FIG. 12 is performed by the processing circuitry 15 reading and executing the computer program corresponding to the control function 152 from the internal storage circuitry 16. At Step S106, the processing circuitry 15 displays the target and the corrected guideline.

As described above, the acquisition function 153 according to the first embodiment acquires the first positional information indicating the position of a puncture needle in the space from which an ultrasonic image is acquired and the second positional information indicating the position of the puncture needle included in the ultrasonic image. The calculation function 154 calculates a bend in the puncture needle based on the first positional information and the second positional information. The correction function 155 corrects the position of information indicating the puncture needle with respect to the ultrasonic image assumed based on the first positional information. With this configuration, the ultrasonic diagnostic apparatus according to the first embodiment can correct the guideline for the puncture needle based on the bend in the puncture needle, thereby improving the workflow of the procedure.

The control function 152 according to the first embodiment defines the distal end of the puncture needle being operated as a viewpoint and defines the direction of movement of the puncture needle as a line-of-sight direction. The control function 152 displays, on the display 2, a display image obtained by arranging the puncture needle after the operation and a region of interest on an image the up-and-down direction and the left-and-right direction of which are determined based on the section received from the ultrasonic probe. With this configuration, the ultrasonic diagnostic apparatus according to the first embodiment enables the operator to instinctively grasp the positional relation between the ultrasonic image and the puncture needle, thereby improving the workflow of the procedure.

The control function 152 according to the first embodiment displays, on the display 2, a three-dimensional image indicating the three-dimensional positional relation between a plurality of puncture needles and the region of interest. With this configuration, the ultrasonic diagnostic apparatus according to the first embodiment can support a procedure using a plurality of puncture needles, thereby improving the workflow of the procedure.

The control function 152 according to the first embodiment outputs notification instructing the correction function 155 to perform correction when the position of information indicating the puncture needle corrected by the correction function 155 is changed. With this configuration, the ultrasonic diagnostic apparatus according to the first embodiment can deal with a bend that occurs in a procedure, thereby improving the workflow of the procedure.

The calculation function 154 according to the first embodiment calculates the curvature of a circle passing through the position indicated by the first positional information and the position indicated by the second positional information as a bend in the puncture needle. With this configuration, the ultrasonic diagnostic apparatus according to the first embodiment can readily estimate the bend in the puncture needle.

### Second Embodiment

The following describes a second embodiment that measures the distance between puncture needles. The second embodiment is different from the first embodiment only in the processing performed by the calculation function 154 and the control function 152. The following mainly describes the processing of these functions.

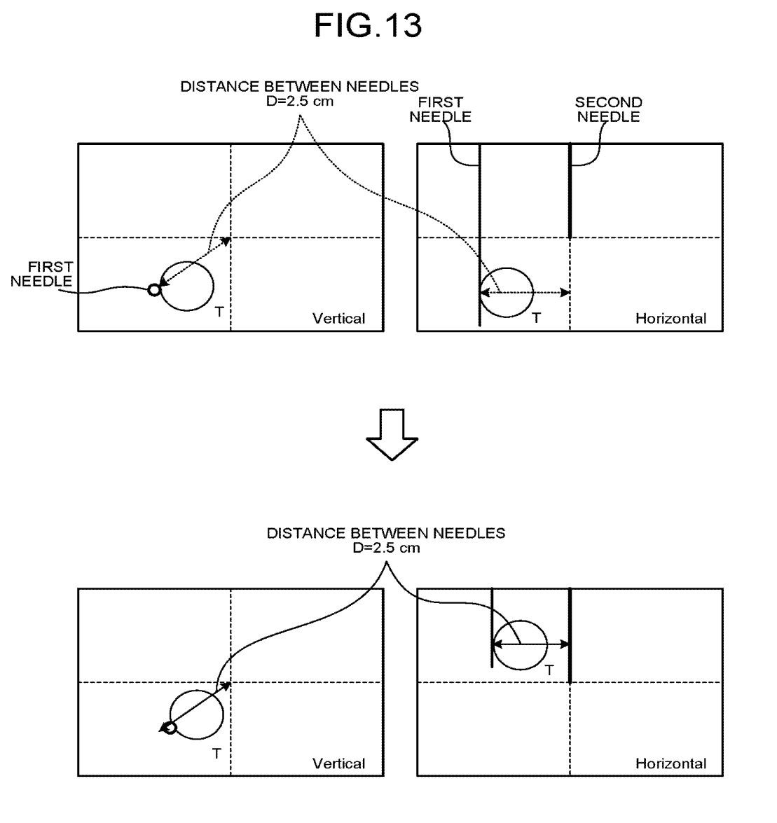

In the ultrasonic diagnostic apparatus according to the second embodiment, the calculation function 154 calculates the distance between a plurality of puncture needles having their positions corrected by the correction function 155. FIG. 13 is a diagram for explaining an example of two-dimensional distance calculation according to the second embodiment. FIG. 13 illustrates a case where an indicator including the “vertical display” and the “horizontal display” is displayed. Specifically, the following describes the second embodiment that calculates the distance between the puncture needles or between the puncture needles and the target two-dimensionally.

When the second puncture needle is being arranged for the target after the first puncture needle is arranged, for example, the calculation function 154 calculates a shortest distance between the puncture needles of “2.5 cm” based on the actual coordinates of the first puncture needle and the coordinates of the needle guide for the second puncture needle as illustrated in the upper figure in FIG. 13. Because the second puncture needle does not reach the position at which the distance between the puncture needles is shortest, the control function 152 displays a line indicating the distance with a dotted line. When the second puncture needle reaches the position at which the distance between the puncture needles is shortest, the control function 152 displays the line indicating the distance with a solid line as illustrated in the lower figure in FIG. 13.

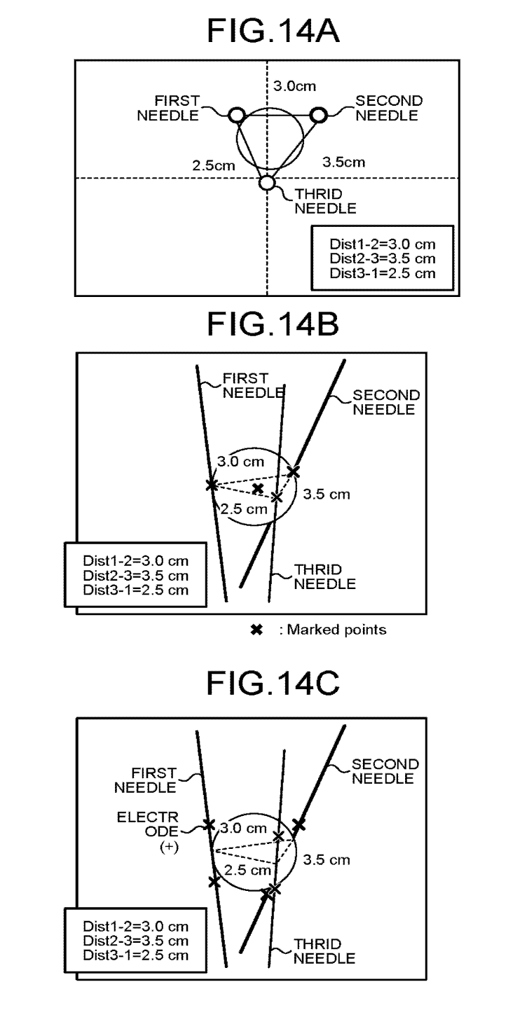

The following describes a case where the distance between puncture needles is displayed three-dimensionally with reference to FIGS. 14A to 14C. FIGS. 14A to 14C are diagrams for explaining an example of three-dimensional distance calculation according to the second embodiment. FIGS. 14A to 14C illustrates a case where three puncture needles are arranged for the target.

When a third puncture needle is being arranged after the first and the second puncture needles are arranged as illustrated in FIG. 14A, for example, the control function 152 displays an indicator of a section perpendicular to the line of the third puncture needle and passing through the center of the target. The calculation function 154 calculates the distance between the puncture needles on the section, and the control function 152 displays the calculated distance on the indicator.

As illustrated in FIG. 14B, the control function 152 can display the positional relation between the first, the second, and the third puncture needles and the target three-dimensionally and display the distance between points marked at desired positions. To display the positional relation three-dimensionally, the control function 152 may display it as a three-dimensional rendering image. Similarly to the three-dimensional indicator described above, the operator can perform rotation in a desired direction, panning, and zooming on the three-dimensional image. The three-dimensional image may be displayed on a stereoscopic three-dimensional monitor.

The calculation function 154 can calculate the distance between desired positions on puncture needles. Puncture needles used for NanoKnife, for example, are each provided with electrodes that supply electricity thereto. The calculation function 154 can calculate the distance of a line connecting the midpoints between the electrodes, for example. As illustrated in FIG. 14C, for example, the control function 152 may display the distances of lines connecting the midpoints between positive electrodes and negative electrodes provided to the respective puncture needles. The positions of the electrodes are determined depending on the types of the needles. The negative electrode is present at a position away from the distal end of the puncture needle by 2 cm, and the positive electrode is present at a position away from the negative electrode by 2 cm, for example. The calculation function 154 acquires information on the types of the currently used puncture needles and calculates the distances of lines connecting the midpoints between the electrodes based on the positions of the electrodes of the acquired types of the needles. The control function 152 displays the calculated distance on the three-dimensional rendering image.

The distance described above may be automatically calculated. The calculation function 154, for example, automatically calculate the distance between the corrected needle guides, and the control function 152 displays the distance between the needles as reference information. Certain points may be specified as automatic measurement points for the distance between the needles. Alternatively, the control function 152 may display a GUI for selecting the types of the needles on the display 2. In this case, the operator selects the types of the needles, whereby the calculation function 154 automatically calculates the distances of lines connecting the midpoints between the electrodes, and the control function 152 displays them. The puncture needles to be a target for calculation of the distance may be optionally selected. Only electrodes that actually supply electricity may be set as a target, for example. By correcting a bend based on the measurement points at which the actual distance between needles is measured (e.g., the midpoints between electrodes), the accuracy in the automatic measurement portions can be increased.

The control function 152 may transmit the value of the distance calculated by the calculation function 154 to the treatment device to set an output value used for treatment. The control function 152 may transmit the calculated value to a Nanoknife device, for example, to determine an appropriate energizing time and output for treating a predetermined range.

If the calculated value is different from a predetermined value, the control function 152 may output a warning. The control function 152, for example, compares the calculated distance with a distance between the puncture needles predetermined in a treatment plan. If the distance between the puncture needles calculated by the calculation function 154 is larger than the distance in the treatment plan, the control function 152 outputs a warning.

While the ultrasonic diagnostic apparatus according to the embodiments above calculates a bend in the puncture needle to correct the needle guide, the embodiments are not limited thereto. The embodiments are also applicable to other modalities, such as X-ray CT apparatuses.

Among the processing contents described in the above-mentioned embodiments, all or part of the processing that is described as being automatically executed can also be manually executed, or all or part of the processing that is described as being manually executed can also be automatically executed by a known method. In addition, the processing procedures, the control procedures, the specific names, and the information including various kinds of data and parameters described herein and illustrated in the accompanying drawings can be arbitrarily changed unless otherwise specified.

The processing method described in the embodiments above is provided by a computer, such as a personal computer and a workstation, executing a processing program prepared in advance. The processing program may be distributed via a network, such as the Internet. The processing program may be recorded in a computer-readable non-transitory recording medium, such as a hard disk, a flexible disk (FD), a compact disc read only memory (CD-ROM), a magneto-optical disc (MO), a digital versatile disc (DVD), and a flash memory including a universal serial bus (USB) memory and an SD card memory. The processing program may be read from the non-transitory recording medium and executed by a computer.

As described above, the embodiments can improve the workflow of the procedure.

While certain embodiments have been described, these embodiments have been presented by way of example only, and are not intended to limit the scope of the inventions.

Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
processing circuitry configured to
acquire first positional information indicating a position of a puncture needle in a space from which an ultrasonic image is acquired and second positional information indicating a position of the puncture needle included in the ultrasonic image;
calculate a bend in the puncture needle based on the first positional information and the second positional information; and
correct a position of information indicating the puncture needle with respect to the ultrasonic image assumed based on the first positional information.

2. The ultrasonic diagnostic apparatus according to claim 1, wherein the processing circuitry is further configured to
display, on a display, information based on the corrected position of information indicating the puncture needle;
calculate a distance between a plurality of puncture needles the position of which is corrected; and
display the calculated distance.

3. The ultrasonic diagnostic apparatus according to claim 2, wherein the processing circuitry is configured to define a distal end of the puncture needle being operated as a viewpoint and define a direction of movement of the puncture needle as a line-of-sight direction and display, on the display, a display image obtained by arranging the puncture needle after operation and a region of interest on an image an up-and-down direction and a left-and-right direction of which are determined based on a section received from an ultrasonic probe.

4. The ultrasonic diagnostic apparatus according to claim 1, wherein the processing circuitry is configured to display, on a display, a three-dimensional image indicating a three-dimensional positional relation between a plurality of puncture needles and a region of interest.

5. The ultrasonic diagnostic apparatus according to claim 1, wherein the processing circuitry is further configured to output notification requesting correction when the corrected position of information indicating the puncture needle is changed.

6. The ultrasonic diagnostic apparatus according to claim 1, wherein the processing circuitry is further configured to
calculate an error between a puncture plan of the puncture needle and an insertion position of the puncture needle; and
output notification requesting correction of the position of the puncture needle when the calculated error exceeds a predetermined threshold.

7. The ultrasonic diagnostic apparatus according to claim 2, wherein the processing circuitry is configured to display, on the display, an output condition for treatment with the puncture needle based on the calculated distance between the puncture needles.

8. The ultrasonic diagnostic apparatus according to claim 1, wherein the processing circuitry is configured to calculate a curvature of a circle passing through the position indicated by the first positional information and the position indicated by the second positional information as the bend in the puncture needle.

9. A medical image diagnostic apparatus comprising:
processing circuitry configured to
acquire first positional information indicating a position of a puncture needle in a space from which a medical image is acquired and second positional information indicating a position of the puncture needle included in the medical image;
calculate a bend in the puncture needle based on the first positional information and the second positional information; and
correct a position of information indicating the puncture needle with respect to the medical image assumed based on the first positional information.
